(19) United States
(12) Patent Application Publication
(10) Pub. No.: US 2015/0064777 A1
(43) Pub. Date: Mar. 5, 2015

(54) APPARATUS FOR DETECTING MICROORGANISMS

(71) Applicants: Jun Hyung Kim, Gwangju (KR); In Chul Park, Gwangju (KR)

(72) Inventors: Jun Hyung Kim, Gwangju (KR); In Chul Park, Gwangju (KR)

(73) Assignee: GLOBAL OPTICAL COMMUNICATION CO., LTD., Gwangju (KR)

(21) Appl. No.: 14/387,649

(22) PCT Filed: Apr. 25, 2012
(86) PCT No.: PCT/KR2012/003168

§ 371 Date: Sep. 24, 2014

(30) Foreign Application Priority Data
Apr. 4, 2012 (KR) 10-2012-0035113

(51) Int. Cl. G01N 33/487; G01N 15/14; C12Q 1/04

(52) U.S. Cl. CPC G01N 33/48735; C12Q 1/04; G01N 15/1404; G01N 2015/0065
USPC 435/288.7

(57) ABSTRACT

Provided is an apparatus for detecting microorganisms, including: a sample accommodation container having an accommodation space in which a measurement sample in which a number of living microorganisms is to be counted is accommodated; an electrode portion installed at the sample accommodation container so as to apply electrical stimulation to the microorganisms of the sample accommodated in the accommodation space; a current applying control portion that is capable of controlling a current applied by the electrode portion; and an image processor that captures an image of the sample accommodated in the sample accommodation container and counts the number of living microorganisms. Accordingly, the number of living microorganisms can be easily identified by electrically stimulating the living microorganisms, and by comparing an image of the living microorganisms generated before electrical stimulation is applied to the living microorganisms with an image of the living microorganisms generated after electrical stimulation is applied to the living microorganisms, the number of living microorganisms can be easily measured.

### TECHNICAL FIELD

The present invention relates to an apparatus for detecting microorganisms, and more particularly, to an apparatus for detecting microorganisms in which a number of living microorganisms can be counted.

### BACKGROUND ART

In general, a number of microorganisms is counted while the microorganisms are viewed with the naked eye through a microscope in a state in which a sample is put in a container, so as to identify the number of microorganisms, and it is identified whether the microorganisms move by physically stimulating them so as to identify living microorganisms among them.

However, in a method of identifying the number of microorganisms by using the microscope, a worker should perform visual identification, and a procedure of stimulating microorganisms is cumbersome, and thus it is very inconvenient to perform an operation of counting the number of living microorganisms.

### DISCLOSURE OF THE INVENTION

### [Technical Problem]

The present invention is directed to providing an apparatus for detecting microorganisms in which an operation of counting a number of living microorganisms can be easily performed by electrically stimulating the living microorganisms.

The present invention is also directed to providing an apparatus for detecting microorganisms in which location movement depending on whether living microorganisms are electrically stimulated can be identified by an image processing method.

### [Technical Solution]

One aspect of the present invention provides an apparatus for detecting microorganisms, including: a sample accommodation container having an accommodation space in which a measurement sample in which a number of living microorganisms is to be counted is accommodated; an electrode portion installed at the sample accommodation container so as to apply electrical stimulation to the microorganisms of the sample accommodated in the accommodation space; a current applying control portion that is capable of controlling a current applied by the electrode portion; and an image processor that captures an image of the sample accommodated in the sample accommodation container and counts the number of living microorganisms.

The accommodation space of the sample accommodation container may be formed to constitute a flow path which extends in a zigzag form and of which one end and the other end are closed and upper part is open, and the electrode portion may include first and second electrodes that respectively extend from one end and the other end of the flow path and are spaced apart from each other.

The accommodation space of the sample accommodation container may be formed to constitute a flow path which extends in a zigzag form and of which one end and the other end are closed and upper part is open, and the electrode portion may include a transparent electrode installed to extend along the flow path and connection terminals that extend from both ends of the transparent electrode toward an outside of the sample accommodation container and are connected to the current applying control portion.

The electrode portion may include: a plurality of horizontal transparent electrodes that are spaced apart from each other in a first direction in the accommodation space of the sample accommodation container; a plurality of vertical transparent electrodes that are spaced apart from each other in a direction perpendicular to the first direction and constitute a matrix shape together with the plurality of horizontal transparent electrodes; and connection terminals that connect the plurality of horizontal transparent electrodes and the plurality of vertical transparent electrodes to the current applying control portion.

The image processor may include: a camera that captures an image of the sample accommodation container; and a vision recognition processor that controls the current applying control portion, compares an image captured by the camera before a current is applied to the sample accommodation container by the current applying control portion with an image captured by the camera after the current is applied to the sample accommodation container by the current applying control portion, counts the number of microorganisms of which locations are changed, and displays the counted number of living microorganisms on a display portion.

### [Advantageous Effects]

As described above, a number of living microorganisms can be easily identified by electrically stimulating the living microorganisms, and by comparing an image of the living microorganisms generated before electrical stimulation is applied to the living microorganisms with an image of the living microorganisms generated after electrical stimulation is applied to the living microorganisms, the number of living microorganisms can be easily measured.

### BRIEF DESCRIPTION OF THE DRAWINGS

FIG. 1 is a side view of an apparatus for detecting microorganisms according to an embodiment of the present invention.

FIG. 2 is a view of a sample accommodation container of FIG. 1.

FIG. 3 is a block diagram of a control system of the apparatus for detecting microorganisms illustrated in FIG. 1.

FIG. 4 is a plan view of a sample accommodation container according to a second embodiment of the present invention.

FIG. 5 is a perspective view of a sample accommodation container according to a third embodiment of the present invention.

### BEST MODE FOR CARRYING OUT THE INVENTION

Hereinafter, an apparatus for detecting microorganisms according to exemplary embodiments of the present invention will be described with reference to the attached drawings in detail.

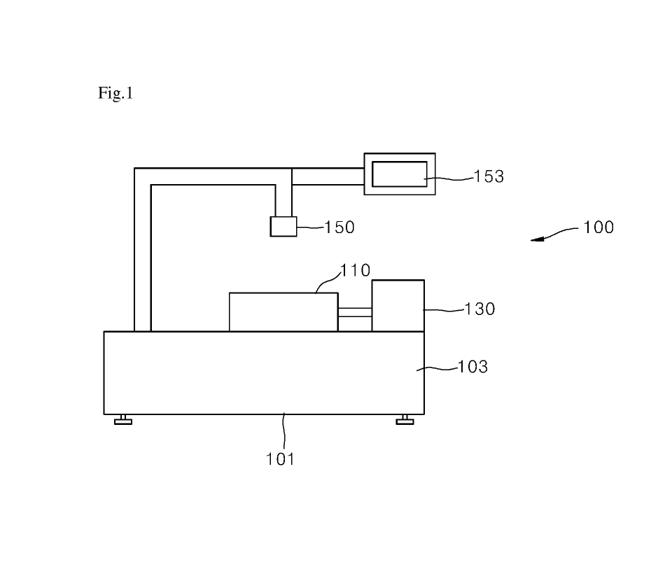

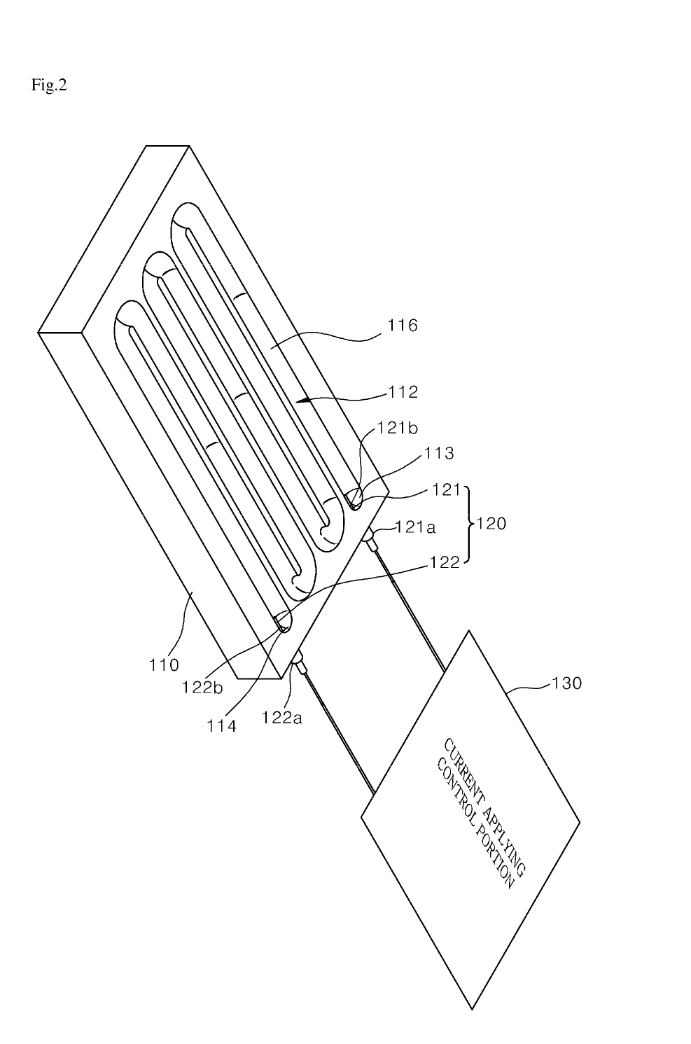

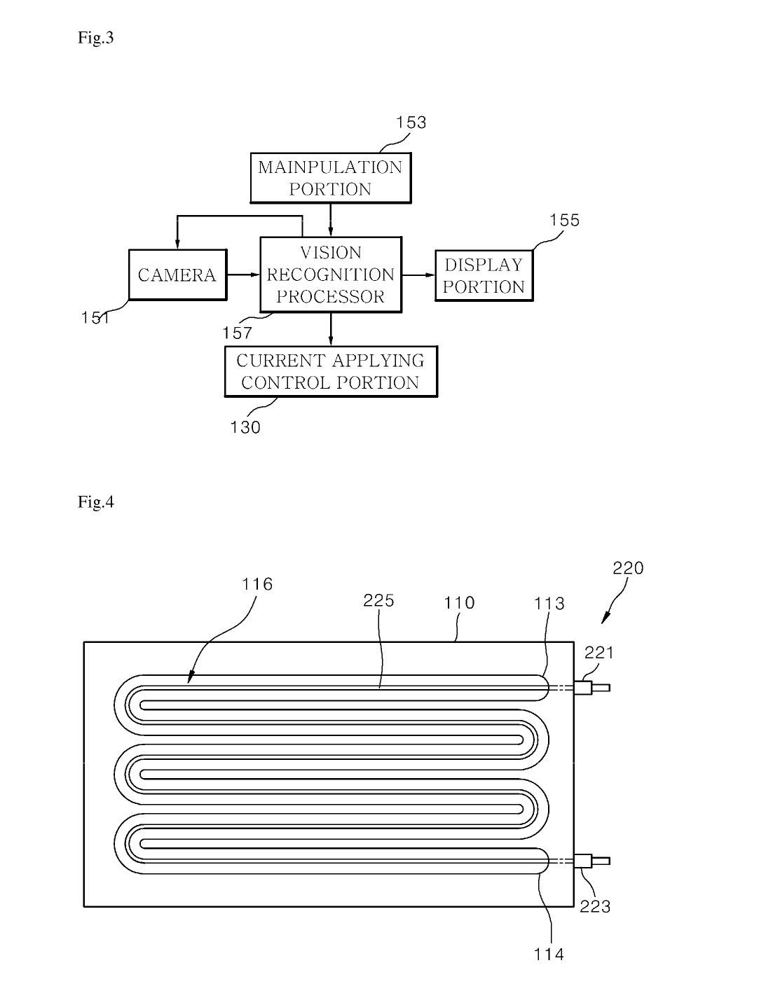

FIG. 1 is a side view of an apparatus for detecting microorganisms according to an embodiment of the present invention, FIG. 2 is a view of a sample accommodation container of FIG. 1, and FIG. 3 is a block diagram of a control system of the apparatus for detecting microorganisms illustrated in FIG. 1.

Referring to FIGS. 1 through 3, an apparatus 100 for detecting microorganisms according to the present invention includes a sample accommodation container 110, an electrode portion 120, a current applying control portion 130, and an image processor 150.

The sample accommodation container 110 has an accommodation space 112, an upper part of which is open, so that a measurement sample in which the number of living microorganisms is to be counted can be accommodated in the sample accommodation container 110.

The accommodation space 112 of the sample accommodation container 110 constitutes a flow path 116 which extends in a zigzag form and in which one end 113 and the other end 114 are closed and an upper part is open.

Here, when the accommodation space 112 of the sample accommodation container 110 constitutes the flow path 116 in the zigzag form, an extension length of the accommodation space 112 of the sample accommodation container 110 may be increased while the width of the flow path 116 is decreased. With respect to flow of the microorganisms, the accommodation space 112 of the sample accommodation container 110 is limited by the width of the flow path 116 in a widthwise direction of the flow path 116 so that flow of the microorganisms in a lengthwise direction of the flow path 116 can be induced and thus efficiency of identifying location movement of the microorganisms depending on whether electrical stimulation is performed, which will be described later, can be improved.

The electrode portion 120 is installed at the sample accommodation container 110 so as to apply electrical stimulation to the microorganism of the same accommodated in the accommodation space 112.

The electrode portion 120 includes first and second electrodes 121 and 122 that respectively extend from one end 113 and the other end 114 of the flow path 116 into the flow path 116.

Here, the first electrode 121 has a first contact portion 121b that protrudes from a first connection terminal 121a that protrudes outward from one end 113 of the flow path 116 of the sample accommodation container 110, into the accommodation space 112 by a predetermined length. Here, the first contact portion 121b of the first electrode 121 may be installed to be exposed in the accommodation space 112 so that the first electrode 121 can maintain contact with the sample put in the accommodation space 112. Alternatively, the first contact portion 121b of the first electrode 121 may be inwardly inserted from one end 113 of the flow path 116 and may be exposed, and when the first contact portion 121b of the first electrode 121 protrudes into the accommodation space 112, a protrusion length of the first contact portion 121b may be properly set.

The second electrode 122 has a second contact portion 122b that protrudes from a second contact terminal 122a that protrudes outward from the other end 114 of the flow path 116 of the sample accommodation container 110, into the accommodation space 112 by a predetermined length.

Here, the first electrode 121 and the second electrode 122 are spaced apart from each other along the flow path 116 and apply electric potentials applied by the current applying control portion 130 that will be described later to the microorganisms through the sample in the flow path 116.

The current applying control portion 130 controls currents applied through the first and second electrodes 121 and 122 of the electrode portion 120.

The current applying control portion 130 is configured to control currents by adjusting the electric potential or impedance applied through the first and second electrodes 121 and 122 by control of the image processor 150 that will be described later.

The image processor 150 captures an image of the sample accommodated in the accommodation space 112 of the sample accommodation container 110, counts the number of living microorganisms and displays the counted number of living microorganisms on a display portion 155.

The image processor 150 includes a camera 151, a manipulation portion 153, the display portion 155, and a vision recognition processor 157.

The camera 151 is installed at a body 101 so as to capture an image of the accommodation space 112 of the sample accommodation container 110 mounted on a base frame 103 of the body 101 in a separate location.

Here, the camera 151 having a magnification at which the microorganisms can be identified may be used.

The manipulation portion 153 includes keys that support manipulations for an operation of counting the number of living microorganisms, such as an execution key for performing an operation of counting the number of microorganisms and a manipulation key for setting a current value to be applied.

The display portion 155 is controlled by the vision recognition processor 157 and displays display information.

The current applying control portion 159 is controlled by the vision recognition processor 157 and controls a current to be applied by the electrode portion 120.

When the execution key is manipulated by the manipulation portion 153, the vision recognition processor 157 stores, in an internal memory, an image generated in a no-current-applied state in which an image of the sample accommodation container 110 is captured by the camera 151 in a state in which no current is applied to the sample accommodation container 110, controls the current applying control portion 130 so that the set current can be applied through the sample accommodation container 110, stores, in the internal memory, an image generated in a current-applied state in which the image of the sample accommodation container 110 is captured by the camera 151, compares the image captured by the camera 151 before a current is applied to the sample accommodation container 110 by the current applying control portion 130 with the image captured by the camera 151 after the current is applied to the sample accommodation container 110 by the current applying control portion 130, counts the number of microorganisms of which locations are changed, and displays the counted number of living microorganisms on the display portion 155.

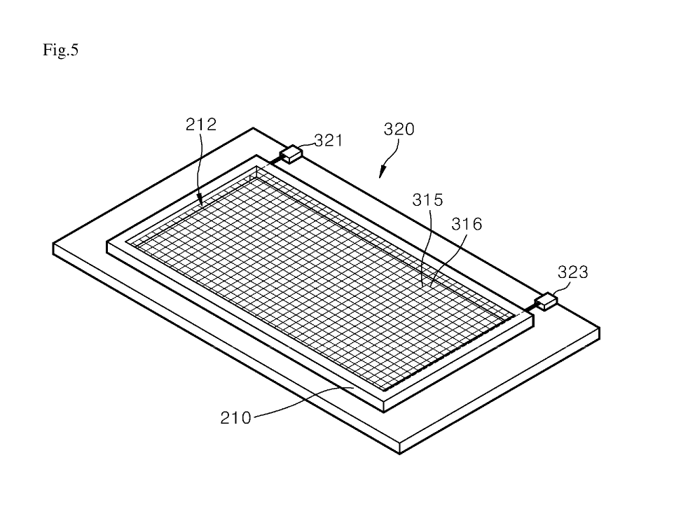

As illustrated in FIG. 4, an electrode portion 220 that is installed at the sample accommodation container 110 and has a structure including a transparent electrode 225 that is installed in a band shape to extend along the flow path 116 formed in the zigzag form and two connection terminals 221 and 223 that extend from both ends of the transparent electrode 225 toward an outside of the sample accommodation container 110 and respectively extend outward from one end 113 and the other end 114 of the flow path 116, so as to be connected to the current applying control portion 130, may be used.

Here, the transparent electrode 225 may be an indium tin oxide (ITO) electrode.

Alternatively, as illustrated in FIG. 5, an electrode portion 320 including a plurality of horizontal transparent electrodes 315a that are spaced apart from each other in a first direction in an accommodation space 212 inserted in a rectangular shape of a sample accommodation container 210, a plurality of vertical transparent electrodes 316 that are spaced apart from each other in a direction perpendicular to the first direction and constitute a matrix shape together with the plurality of horizontal transparent electrodes 315a and connection terminals 321 and 323 that connect the plurality of horizontal transparent electrodes 315a and the plurality of vertical transparent electrodes 316 to the current applying control portion 130 may be used.

Here, portions at which the horizontal transparent electrodes 315 and the vertical transparent electrodes 316 cross one another, are joined to each other so that currents can flow through the portions.

When the apparatus 100 for detecting microorganisms operates after a measurement sample, for example, sea water, is injected into the sample accommodation container 110 or 210, the number of living microorganisms can be easily measured using information regarding a change in locations of the microorganisms before electrical stimulation is applied to the microorganisms and locations of the microorganisms after electrical stimulation is applied to the microorganisms, through the above-described procedures.

While the invention has been shown and described with reference to certain exemplary embodiments thereof, it will be understood by those skilled in the art that various changes in form and details may be made therein without departing from the spirit and scope of the invention as defined by the appended claims.

1. An apparatus for detecting microorganisms, comprising:
a sample accommodation container having an accommodation space in which a measurement sample in which a number of living microorganisms is to be counted is accommodated;
an electrode portion installed at the sample accommodation container so as to apply electrical stimulation to the microorganisms of the sample accommodated in the accommodation space;
a current applying control portion that is capable of controlling a current applied by the electrode portion; and
an image processor that captures an image of the sample accommodated in the sample accommodation container and counts the number of living microorganisms.

2. The apparatus of claim 1, wherein the accommodation space of the sample accommodation container is formed to constitute a flow path which extends in a zigzag form and in which one end and the other end are closed and an upper part is open, and
the electrode portion comprises first and second electrodes that respectively extend from the one end and the other end of the flow path and are spaced apart from each other.

3. The apparatus of claim 1, wherein the accommodation space of the sample accommodation container is formed to constitute a flow path which extends in a zigzag form and in which one end and the other end are closed and an upper part is open, and
the electrode portion comprises a transparent electrode installed to extend along the flow path and connection terminals that extend from both ends of the transparent electrode toward an outside of the sample accommodation container and are connected to the current applying control portion.

4. The apparatus of claim 1, wherein the electrode portion comprises:
a plurality of horizontal transparent electrodes that are spaced apart from each other in a first direction in the accommodation space of the sample accommodation container;
a plurality of vertical transparent electrodes that are spaced apart from each other in a direction perpendicular to the first direction and constitute a matrix shape together with the plurality of horizontal transparent electrodes; and
connection terminals that connect the plurality of horizontal transparent electrodes and the plurality of vertical transparent electrodes to the current applying control portion

5. The apparatus of claim 2, wherein the image processor comprises:
a camera that captures an image of the sample accommodation container; and
a vision recognition processor that controls the current applying control portion, compares an image captured by the camera before a current is applied to the sample accommodation container by the current applying control portion with an image captured by the camera after the current is applied to the sample accommodation container by the current applying control portion, counts the number of microorganisms of which locations are changed, and displays the counted number of living microorganisms on a display portion.

6. The apparatus of claim 3, wherein the image processor comprises:
a camera that captures an image of the sample accommodation container; and
a vision recognition processor that controls the current applying control portion, compares an image captured by the camera before a current is applied to the sample accommodation container by the current applying control portion with an image captured by the camera after the current is applied to the sample accommodation container by the current applying control portion, counts the number of microorganisms of which locations are changed, and displays the counted number of living microorganisms on a display portion.

7. The apparatus of claim 4, wherein the image processor comprises:
a camera that captures an image of the sample accommodation container; and
a vision recognition processor that controls the current applying control portion, compares an image captured by the camera before a current is applied to the sample accommodation container by the current applying control portion with an image captured by the camera after the current is applied to the sample accommodation container by the current applying control portion, counts the number of microorganisms of which locations are changed, and displays the counted number of living microorganisms on a display portion.
